(19) United States
(12) Patent Application Publication
(10) Pub. No.: US 2016/0000985 A1
(43) Pub. Date: Jan. 7, 2016

(54) EXTRAVASCULAR DEVICES SUPPORTING AN ARTERIOVENOUS FISTULA

(71) Applicant: Abbott Cardiovascular Systems Inc., Santa Clara, CA (US)

(72) Inventors: Paul Consigny, San Jose, CA (US); Erik Eli, Redwood City, CA (US); Stephen D. Pacetti, San Jose, CA (US); Mikael Trollsas, San Jose, CA (US); John Stankus, Campbell, CA (US)

(21) Appl. No.: 14/322,826

(22) Filed: Jul. 2, 2014

(51) Int. Cl. A61M 1/30

(52) U.S. Cl. CPC A61M 1/301

(57) ABSTRACT

A medical device includes a curved tubular body configured for being used as an extravascular device to support vein maturation following the formation of an arteriovenous fistula. The tubular body is curved. The tubular body has an entrance angle of less than about 40 degrees to improve blood flow from the artery into the vein. And the tubular body includes a cuff or edge at the proximal end to stabilize the tubular body at the fistula.

### FIELD OF THE INVENTION

The present invention relates to implantable medical devices associated with the creation of, and/or the maturation of an arteriovenous (AV) fistula access structure for hemodialysis.

### BACKGROUND OF THE INVENTION

AV Fistula (a connection between an artery and a vein) are a desired access structure for the dialysis of kidney failure patients. FIG. 1 illustrates a matured portion of the vein near the artery, which acts as a re-usable cannula access site proximal the AV fistula.

About 42% of surgically created AV Fistula fail to mature; that is, the portion of the vein proximal the fistula fails to adapt physiologically to accommodate the higher arterial pressure. When this venous portion (or side of the AV fistula) matures, it becomes usable as a cannula access site for dialysis (FIG. 1). Maturation can take about 6 weeks from surgically forming the fistula. Failure to mature and/or act as a good dialysis access site is most commonly the result of poor blood flow (low blood pressure/low blood flow rates) in the venous portion of the fistula. About 74% of these failures are salvaged by some form of intervention, followed by maturation of the venous side in another 6-8 weeks. The remaining about 11% of the cases are regarded as failures, which necessitates creating an AV Fistula at another site. The most common site of initial AV Fistula creation is the fore arm. If a new AV Fistula is required, a new site proximal of the previous/failed site is chosen. Typically, there are 3 potential sites per arm.

Patients without a mature AV Fistula require some other, less desirable form of dialysis access for the standard 3 times a week dialysis regimen until a mature fistula is available. Additionally, about a third of mature fistula fail in a year. The health of kidney failure patients without a functioning mature AV Fistula deteriorates at a more rapid rate than those with one. Deteriorating health makes the subsequent creation of a functioning mature AV Fistula less probable, necessitating a significant number of interventions or access procedures resulting in poorer survival rates. Thus, a significant number of interventions and procedures may be avoided or significantly delayed, significant cost savings realized and the survival rate of dialysis patients significantly improved by decreasing the failure to mature rate of newly created AV fistula and by reducing the rate at which mature fistula fail.

There is evidence that the shape of an arteriovenous fistula can affect long term durability. For example, Papachristou (2012) and Krishnamoorthy (2012) have indicated that a curved fistula is preferred to a straight fistula because the curved fistula results in greater flow rates, lesser differences in wall shear stress, greater venous dilatation, and less eccentric neointimal hyperplasia. Papachristou E and Vazquez-Padron R I. From basic anatomic configuration to maturation success. Kidney International 81: 724-726, 2012. Krishnamoorthy M K, Banerjee R K, Wang Y et al. Anatomic configuration affects the flow rate and diameter of porcine arteriovenous fistula. Kidney International 81: 745-750, 2012. In addition, Ene-lordache B et al (2013) have found that angle at the origin of a “side-to-end” arteriovenous fistula is very important. Their research indicates that an angle of 30 degrees is preferred over angles of 45, 60 or 90 degrees. Ene-lordache B, Cattaneo L, Dubini G, Remuzzi A. Effect of anastomosis angle on the localization of disturbed flow in “side-to-end” fistula for haemodialysis access. Nephrol Dial Transplant 28: 997-1005, 2013.

There are no known extravascular or perivascular devices available that can effectively and reliably assist a surgeon in maintaining a more desirable AV fistula construct. Accordingly, there is a need for a device that can aid in creating the correct anatomy by providing the appropriate support in the appropriate locations and in the appropriate configurations that promote long-term arteriovenous (AV) fistula patency.

### SUMMARY OF THE INVENTION

The invention provides an extravascular or perivascular arteriovenous (AV) guide intended for being placed at an anastomosis to support and help achieve vein maturation. The apparatus is intended for being placed at an anastomosis to support and help achieve a vein maturation including an about 15 to 45 degrees, preferably about 30 degree, take-off angle (or less than about 45 degrees) between the vein and artery at the fistula. The apparatus is placed when the anastomosis is made and remains to help produce an about 30 take-off angle for the matured vein.

U.S. application Ser. No. 14/063,984 (attorney docket: 62571.770) (“'984 application”) disclose extravascular wraps for an AV fistula. Discussed therein are take-off angles for the venous portion of the fistula. The '984 application proposes an obtuse take angle, which is measured with respect to axis A in FIG. 2A of the '984 application, and a curved venous support portion for an AV guide. The present disclosure includes embodiments directed to an extravascular AV guide that instead provide an acute take-off angle and the venous support portion is comparatively straight or devoid of a curvature as described in the '984 application. U.S. application Ser. No. 14/253,719 (attorney docket: 62571.889) (“'719 application”) discloses embodiments of stents or support devices intraluminally placed at a fistula to support the same range of take-off angles as embodiments of an AV wrap disclosed herein.

The invention, in one aspect, is directed to a medical device supporting a desired venous take off angle θ of about 5, 10, 15, 20, 25, less than about 30 degrees, between about 20-45 degrees or between about 15 to 45 degrees relative to axis A in FIG. 2A. This angle helps decrease failure to mature rates. Take-off angles above about 45 degrees (relative to the artery longitudinal axis), have been associated with low flow of the fistula. Loss of (or poor) patency of the attachment site is associated with low flow and eventual failure of the fistula.

According to some embodiments, the AV guide is sized to initially fit at least a vein portion distal of the fistula loosely and has an internal diameter in the range of 4-8 mm. A variety of sizes would be available depending on the patient anatomy. For example, an average outer diameter of a vein portion of the fistula is roughly 6 mm, so the AV guide would have an ID of 6 mm. According to some embodiments, the AV guide includes bioresorbable or non-bioresorbable wraps, cuffs or shells, respectively, which allow a surgeon to easily fit or place the wrap at the fistula. A tubular body is formed, according to some embodiments. In other embodiments a wire frame is made. In the case of a tubular body, the structure may be made from bioresorbable threads combined with a shell so that a channel or opening circumscribing the vein will easily open as the vein matures and radially presses outward. The venous portion of the AV guide may have a diameter of 4-10 mm and a length of 5-10 cm.

In accordance with the foregoing, there is an AV stent or scaffold, medical device, method for making such an AV stent or scaffold, a method of using an AV stent or scaffold, or method for assembly of a medical device comprising such a AV stent or scaffold, and/or a medical device comprising a balloon, having one or more, or any combination of the following things (1)-(35):
(1) A take-off angle θ of about 30, or between about 15-45 degrees, or less than about 45 degrees.
(2) A shell for supporting a take-off angle for the venous portion of an AV fistula.
(3) The shell is a clamshell having a hinge portion, e.g., living hinge, and half sections brought together to form a body surrounding an AV fistula.
(4) The shell is made from a porous polymer material.
(5) The shell is made from a durable or biodegradable polymer, or a combination thereof.
(6) A arterial shell portion is formed by two half-cylinders.
(7) The shell has a venous portion comprised of half cylinders, or two halves of a frustum, or two halves of a tubular body having a first diameter (D1) proximal the fistula location and a second diameter (D2) distal of the fistula location, where D2 is greater than D1, e.g., D2 is a factor 2, 4 or 6 greater than D1.
(8) The arterial portion of the shell has an average lumen size that is greater than an average lumen size of the venous portion. The average lumen sizes can be a diameter.
(9) The shell has a diameter that increases over time, or a reducing radial stiffness over time as a bioresorbable portion of the shell losses strength. The time period being 6-12 weeks from implanting device at the fistula.
(10) The shell includes magnets for holding edges together.
(11) The shell is drug coated to prevent cell infiltration or control neointimal hyperplasia.
(12) The shell elutes a vasodilator that promotes venous dilation.
(13) The shell includes agents that degrade the external structure of the vessel to permit expansion.
(14) At least a portion of the shell is configured to bio-resorb or bio-degrade within 6-12 weeks of being implanted in the body, such that there is a significant loss of mechanical strength or stiffness for any portion of the shell surrounding or in contact with a venous portion of the fistula.
(15) A method for supporting a take-off angle for a venous portion of a fistula including placing the shell at the fistula.
(16) A moldable sheet of material for supporting a take-off angle of a fistula. The sheet achieves cross-linking (to form a permanent shape) by moisture cure, temperature, UV curing etc. The material may be permanent or durable, or the material may be bioresorbable. Once shaped and placed around the vasculature, the ends may be held together by magnets, as disclosed earlier.
(17) Components A and B for crosslinking.
(18) A moldable sheet that is a thermoset, moisture activated or thermoplastic material.
(19) A method for placing a wrap at an AV fistula including molding the sheet according to a patient's vasculature.
(20) A clip that can be wire formed and made from an elastic material.
(21) One or more springs for supporting a vessel and maintaining a take-off angle form a fistula.
(22) A plurality of clips connected by a member including a wire and arranged so that a first clip is orientated at the take-off angle with respect to at least one other clip.
(23) The clips and/or member are made from elliptical, e.g., circular, or flat wire.
(24) The member has an arcuate portion connecting straight portions thereof.
(25) An apparatus, comprising: a first tubular member having ends; and a second tubular member connected to the first tubular member between the ends and extending from the second tubular member at a take-off angle θ; wherein θ is less than about π/4 radians.
(26) The aspects of disclosure as set forth in (25) in combination with one of, more than one of, or any combination of the following list of things: wherein the apparatus is an extravascular arteriovenous (AV) wrap configured for supporting the venous portion of the fistula at the take-off angle; wherein the AV wrap comprises a porous body made from a biocompatible polymer, polymer blend, metal, metal alloy, or a combination thereof; wherein at least the second tubular member comprises both durable and biodegradable/bioresorbable polymers; and/or wherein the second tubular member comprises durable polymer filaments and woven biodegradable or bioresorbable threads; wherein the apparatus is a shell, the shell comprising: a first part including a first edge, a second part including a second edge, and a hinge portion connecting the first and second parts, wherein the edges are joined by a fastener; wherein the fastener is a magnet; wherein the hinge is a living hinge; wherein the apparatus comprises a porous body comprising a biocompatible polymer and/or metal; wherein the second tubular member is cylindrical or frustoconical; wherein the second tubular member has a first opening proximal the connection to the first tubular member and a second opening distal therefrom, wherein the second opening is larger than the first opening; wherein the second tubular member has a lower surface that forms with a lower surface of the first tubular member an angle of (180−θ) degrees, and the second tubular member has an upper surface that forms with an upper surface of the first tubular member at an angle of less than or equal to θ; wherein an average lumen size for the first tubular member is greater than an average lumen size for the second tubular member and/or one or more of a), b), c) and d):
   a) an agent for preventing cell infiltration or to control neointimal hyperplasia, the agent being selected from the set consisting of everolimus, zotarolimus, ABT-578, sirolimus, umirolimus, biolimus, merilumus, myolimus, novolimus, temsirolimus, deforolimus, and AP23573;
   b) an agent to encourage cell infiltration or to integrate a blood vessel with the AV wrap, the agent being selected from the set consisting of fibroblastic growth factor (FGF), basic FGF, platelet derived growth factor (PDGF), insulin like growth factor 1 (IGF-1), epidermal growth factor (EGF), granulocyte macrophage colony stimulating factor (GMCSF), human growth hormone (HGH), IL-1, TGF-B, and matrix metalloproteinases;
   c) a vasodilator that promotes venous dilation, the vasodilator being selected from the set consisting of a nitric oxide donor, nitroglycerine, papaverine, calcium channel blocker, adenosine, prostacyclin, epinephrine, prostaglandin, L-arginine, bradykinin, natriuretic peptides, alpha blockers, adenocard, sodium nitroprusside, and matrix metalloproteases; and/or
   d) an agent that degrades external structure of a vessel circumscribed by the AV wrap, the agent being selected from the set consisting of collagenases, elastases, metalloproteinases; agents that promote inflammation that results in the release of factors the promote vessel expansion, such as interleukins 1, and TNF-alpha; and agents that block the effects of anti-inflammatory agents, such as blockers of IL-4, IL-10, IL-13, which are anti-inflammatory cytokines.
(29) An apparatus adapted for being molded into an extravascular wrap for supporting a take-off angle of a venous portion of an arteriovenous (AV) fistula, comprising: a sheet of moldable material; wherein the sheet comprises one of a thermoset, moisture-activated or thermoplastic material.
(30) The aspects of disclosure as set forth in (29) in combination with one of, more than one of, or any combination of the following list of things: wherein the sheet comprises one of a backbone material, and connected to the backbone material is material comprising a first group and a second group of material that chemically react with each other to form the wrap; and/or wherein the backbone material is a biodegradable or durable polymer; wherein the first and second groups are selected from the set consisting of one of the pairs of Group A and Group B material in TABLE 2.
(31) An arteriovenous (AV) guide, comprising: a clip defining a first tubular space having a first bore axis; and a member connecting the clip to at least one tubular body defining a second bore axis such that the first bore axis is oriented at an angle of θ or (180−θ) with respect to the second bore axis; wherein θ is less than about π/4 radians.
(32) The aspects of disclosure as set forth in (31) in combination with one of, more than one of, or any combination of the following list of things: wherein the at least one tubular body is a second clip; a pair of curved portions movable between an open position and a closed portion by deformation of a deflection portion interconnecting the curved portions, wherein when in the closed position the pair of curved portions surround the first tubular space; wherein the clip is formed by a first and second u-shaped wire welded to a serpentine wire, the serpentine wire comprising the deflection portion; wherein the first tubular space is larger than a second tubular space circumscribed by the tubular body.
(33) A method for supporting an AV fistula, comprising: molding a sheet into an AV wrap; and placing the wrap around the AV fistula.
(34) A method for supporting an AV fistula using an AV guide, including placing the tubular body over a vein portion and attaching the clip to an artery portion of the fistula.
(35) A method for supporting an AV fistula using an AV wrap.

### INCORPORATION BY REFERENCE

All publications and patent applications mentioned in the present specification are herein incorporated by reference to the same extent as if each individual publication or patent application was specifically and individually indicated to be incorporated by reference. To the extent there are any inconsistent usages of words and/or phrases between an incorporated publication or patent and the present specification, these words and/or phrases will have a meaning that is consistent with the manner in which they are used in the present specification.

### BRIEF DESCRIPTION OF THE DRAWINGS

FIG. 1 is a side-view of the arm of a patient receiving dialysis. A fistula is shown.

FIG. 2A shows a side-view of an arteriovenous (AV) wrap or shell placed over a fistula.

FIG. 2B shows a top view of the shell of FIG. 2A (without artery or vein included) as taken from Section IIB-IIB in FIG. 2A.

FIG. 2C shows a plan view of the shell of FIG. 2A when opened, and as taken from Section IIC-IIC in FIG. 3A (artery and vein not shown).

FIGS. 3A and 3B show steps for placing the shell of FIG. 2A over an artery and vein portion of the fistula.

FIGS. 4A, 4B, 4C and 4D summarize the time course of fistula maturation. FIGS. 4A and 4B illustrate the blood flow rate and change in diameter for a successful AV fistula. FIGS. 4C and 4D illustrate the blood flow rate and change in diameter for a failing AV fistula.

FIG. 5 is a perspective view of an AV fistula support device utilizing interconnected clips for supporting the take-off angle for the venous portion of the fistula.

FIG. 5A is a perspective view of a wire of FIG. 5 interconnecting the clips of the device ion FIG. 5

FIG. 6A is a first perspective view of a clip used in the device of FIG. 5.

FIG. 6B is a second perspective view of the clip of FIG. 6A, with the clip configured in an open position for placing around a vessel.

FIG. 6C is a side view of the clip of FIG. 6A.

FIG. 6D is a side view illustrating how the clip of FIG. 6A is placed around a vessel.

FIG. 6E is a top view of the clip of FIG. 6A taken at section VIC-VIC.

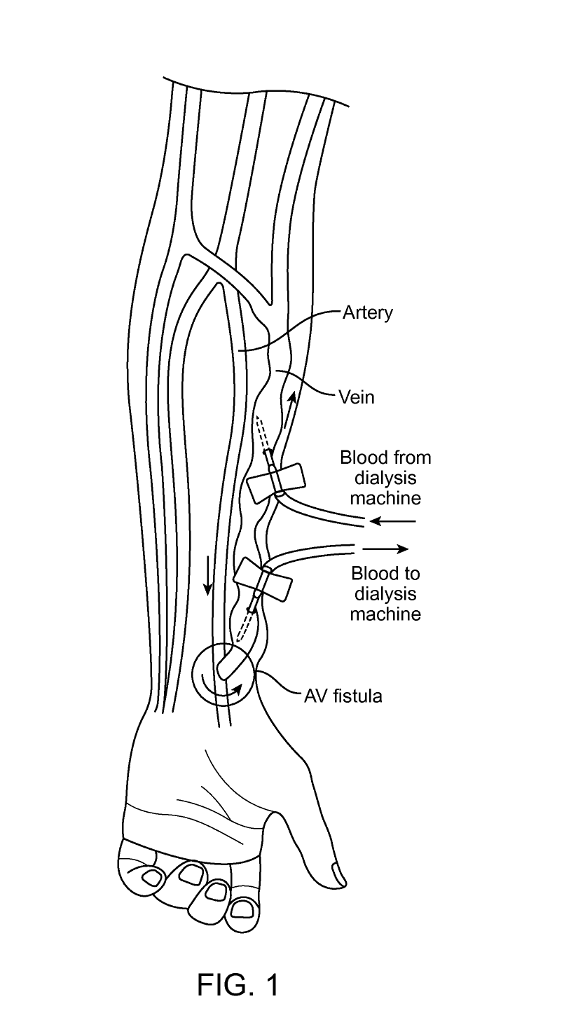

### DETAILED DESCRIPTION OF EMBODIMENTS

For purposes of this disclosure, the following terms and definitions apply:

When referring to a vein or artery prior to making a fistula, a “proximal end” refers to an end closest to the torso of the body, whereas a “distal end” refers to the end furthest from the torso of the body. In contrast, after the fistula is made, or when referring to a medical device's intended location relative to a fistula or anastomosis, the terms “proximal” and “distal” are instead intended to be made with respect to the relative location of the fistula or anastomosis. Thus, for example, the end of a AV Guide closest to the fistula will be called the “proximal” end and the end furthest from the fistula the “distal” end. Thus, generally speaking, prior to making the fistula the former terminology is used. And after the fistula is made “proximal” and “distal” always refers to a location relative to the fistula.

The terms “anastomosis” and “fistula” may be used interchangeably in this description. For purposes of the disclosure the two terms mean the same thing and refer to the arteriovenous (AV) type of anastomosis or fistula.

The terms “about” or “approximately” mean 30%, 20%, 15%, 10%, 5%, 4%, 3%, 2%, 1.5%, 1%, between 1-2%, 1-3%, 1-5%, or 0.5%-5% less or more than, less than, or more than a stated value, a range or each endpoint of a stated range, or a one-sigma, two-sigma, three-sigma variation from a stated mean or expected value (Gaussian distribution). For example, d1 about d2 means d1 is 30%, 20%, 15%, 10%, 5%, 4%, 3%, 2%, 1.5%, 1%, 0%, or between 1-2%, 1-3%, 1-5%, or 0.5%-5% different from d2. If d1 is a mean value, then d2 is about d1 means d2 is within a one-sigma, two-sigma, or three-sigma variance from d1.

It is understood that any numerical value, range, or either range endpoint (including, e.g., “approximately none”, “about none”, “about all”, etc.) preceded by the word “about,” “substantially” or “approximately” in this disclosure also describes or discloses the same numerical value, range, or either range endpoint not preceded by the word “about,” “substantially” or “approximately.”

A procedure for forming an AV fistula is explained in the documents incorporated by reference herein. As noted therein, after the fistula is formed, there is no guarantee that the vein will retain a desirable flow facilitating curve. An AV stent or scaffold according to the disclosure helps to maintain a desired venous shape to increase the patency period for the fistula. Importantly, the devices disclosed herein can promote increased flow rate through the fistula by affecting the flow characteristics/patterns such that there are no regions of low wall shear stress and/or less circular/stagnant flow along in the vein wall, which helps prevent a stenosis from forming at the fistula or adjacent portions of the vein. Preferably the AV stent or scaffold (or combination thereof) is such that it causes the vein to mature into a shape producing a relatively low acceleration (rate of direction change) of the flow as it is diverted from the artery to vein. Moreover, the shape minimizes or eliminates stagnant or circular blood flow and avoids the forming of low flow regions that result in minimal or no shear stress along the vessel walls. Dimensional goals for the fistula are to enlarge to a diameter on the order of 6 mm and lie no more than 6 mm beneath the skin surface.

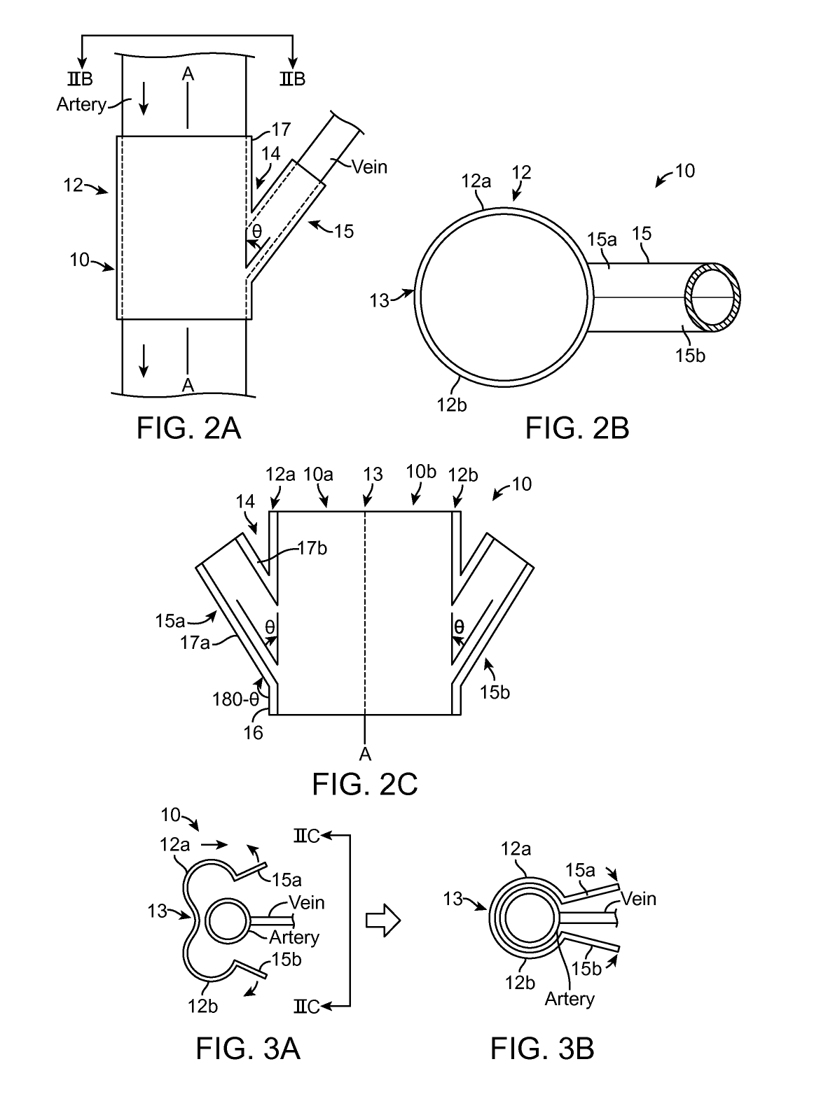

Referring to FIGS. 2A-2C there is shown an AV guide according to a first aspect of the disclosure: a support structure resembling a clam shell. The claim shell 10 is configured to have a vein support portion 15 and artery support portion 12. Referring to FIG. 2A, the shell 10 has been placed at the fistula such that the vein support 15 supports and maintains the vein at the take-off angle θ at least for the first 6-12 weeks following formation of the fistula. The illustrated take-off angle is acute with respect to axis A. The shell 10 is positioned so that a mean blood flow direction changes by 180−θ degrees when passing from the artery to the vein.

Referring to FIGS. 3A-3B there are depicted steps for placing the shell 10 over the artery and vein to arrive at the shell 10 shown in FIG. 2A. As can be appreciated from these views, the shell 10 is configured to have a flexible hinge 13 so that the shell 10 can be opened, placed around the artery and vein, then closed to surround vein and artery portions, as depicted in FIG. 2A. FIG. 2B shows a top view taken at IIB-IIB of FIG. 2A, but with the vein and artery from FIG. 2A removed in FIG. 2B.

Referring to FIG. 2C, there is shown a view of the shell 10 taken at IIC-IIC in FIG. 3A. Here is shown two halves 10a, 10b of the shell 10 joined by a hinge portion 13, which in the illustrated embodiment is a living hinge. The halves 10a, 10b are mirror-images of each other or symmetric about the axis A. As can be appreciated from this view, FIGS. 3A-3B, and FIG. 2B the vein portion 15 is formed by portions 15a, 15b and the artery portion 12 is formed by portions 12a, 12b. The following description refers to the left-side half 10a in FIG. 2C with the understanding that the same or similar description applies to the right-side half 10b.

The half-tube vein portion 15a extends from the artery portion 12a at the angle θ, with respect to axis A; or a lower edge 17a of the portion 15a forms an angle of 180−θ with respect to a lower edge 16 of the artery portion 12a. In some embodiments the half-tube portion 15a is a half-cylinder extension of the half cylinder portion 12a. In alternative embodiments the portion 15a can be a half-frustum or form one hall of a portion 15a having a frustoconical shape whereby the smaller opening of the frustum is proximal the fistula and the larger opening distal of the fistula. In another embodiment the body shape for portion 15a has a first portion proximal the fistula that is constant in diameter (as illustrated) then flares out towards the distal end. The increased diameter provided in the alternative embodiments provides the space to allow the vein to increase in diameter while providing the structure proximal the fistula to support the take-off angle and proper vein maturation. The edge 17a can be straight so that the angle 180−θ is constant from the proximal to distal ends of the portion 15a; however, the corresponding edges forming the angle at 14 would decrease when moving from the edge portions proximal the fistula to distal the fistula.

In the foregoing embodiments of the cylindrical, or frusto-conical shapes of the portion 15, i.e., portions 15a and 15b combined, it will be appreciated that only the inner surface of the lower edge 17 may be needed to maintain the take-off angle by contact with the abluminal surface, just as the tongue structure described in the '719 application (intraluminal AV support) may be all that is needed to maintain the take-off angle from the luminal surface of the vessel. As such, the portion 15 can be shaped to have a taper or flared proximal end, or otherwise have an opening at the distal end that is larger than the opening at the proximal end, e.g., larger by a factor of 2, 4 or 6. This structure type helps avoid adversely interfering with the vein's maturation in response to increased blood flow through the fistula. Alternatively or in addition, in some embodiments upper edges 17b of portion 15 are not connected to each other, so as to reduce or avoid any resistance to the vein increasing in diameter during maturation. The slit or edge surfaces may be formed to have a zig-zag or wavy pattern as shown in the '984 application when the shell 10 is placed around the fistula.

Thus, the shell 10 when closed may provide sufficient space for the vein to expand during the remodeling process while maintaining the take-off angle. This is important as the vein portion of the fistula undergoes considerable positive remodeling and greatly enlarges in size. The venous portion of the shell 10 should have enough space to allow this enlargement. Or the portion 15 should be able to easily dilate if it is close fitting when implanted.

The space between the shell 10 and vessel abluminal surfaces may be filled with a hydrogel, which could be high molecular weight, shear-thinning, pre-crosslinked particles, or in-situ gelling. Examples include Healon (hyaluronic acid), PEG, two component glues such as fibrin glue, Bioglue, two-component PEG, Restylane type of particles, UV-curing, or Pluronics containing free drug, or microparticles or nanoparticles that elute drug. The filling may be used as a medium/vehicle to aid in the retention of drug-eluting particles at the therapy site.

The shell 10 can be made of either porous or nonporous materials. It is believed that a porous structure would be preferable for biocompatibility, tissue vitality, and tissue ingrowth. However, a monolithic or less permeable structure is possible.

Materials to accomplish this include: polyethylene terephthalate (e.g., DACRON), silicone, polyurethanes, polypropylene, polyesters, Pebax, silicones, polyisobutylene and ethylene-alphaolefin copolymers, acrylic polymers and copolymers, vinyl halide polymers and copolymers, poly(vinyl chloride), poly(vinyl fluoride), poly(vinylidene fluoride) (PVDF), poly(vinylidene chloride), poly(vinylidene fluoride-co-hexafluoropropylene) (PVDF-HFP), poly(tetrafluoroethylene-co-vinylidene fluoride-co-hexafluoropropylene), polyvinyl ethers, such as polyvinyl methyl ether, polyacrylonitrile, polyvinyl ketones, polyvinyl aromatics, such as polystyrene, polyvinyl esters, such as poly(vinyl acetate), copolymers of vinyl monomers with each other and olefins, such as ethylene-methyl methacrylate copolymers, acrylonitrile-styrene copolymers, ABS resins, and poly(ethylene-vinyl acetate) copolymers, polyamides, such as Nylon 66 and polycaprolactam, alkyd resins, polycarbonates, polyoxymethylenes, polyimides, polyethers, poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, poly(vinyl butyral), poly(ether urethane), poly(ester urethane), poly(urea urethane), poly(silicone urethane), polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, poly(ether ester), polyalkylene oxalates, polyphosphazenes, poly(fluorophosphazene), poly(phosphoryl choline methacrylate), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, poly(styrene-isoprene-styrene)-PEG (SIS-PEG), polystyrene-PEG, polyisobutylene-PEG, poly(methyl methacrylate)-PEG (PMMA-PEG), polydimethylsiloxane-co-PEG (PDMS-PEG), nitinol, and/or elgiloy.

The shell 10 can also be drug coated in order to prevent cell infiltration or control neointimal hyperplasia. Drugs to accomplish this include: everolimus, zotarolimus, ABT-578, sirolimus, umirolimus, biolimus, merilumus, myolimus, novolimus, temsirolimus, deforolimus, and AP23573. Alternatively, in other embodiments the shell 10 can be drug coated to encourage cell infiltration or to integrate the cover with the vein and/or arterialize it. Drugs for this include mitogens such as fibroblastic growth factor (FGF), basic FGF, platelet derived growth factor (PDGF), insulin like growth factor 1 (IGF-1), epidermal growth factor (EGF), granulocyte macrophage colony stimulating factor (GMCSF), human growth hormone (HGH), IL-1, TGF-B, and matrix metalloproteinases.

The shell 10 may also elute a vasodilator that promotes venous dilation. Possible drugs include a nitric oxide donor, nitroglycerine, papaverine, calcium channel blocker, adenosine, prostacyclin, epinephrine, prostaglandin, L-arginine, bradykinin, natriuretic peptides, alpha blockers, adenocard, sodium nitroprusside, or matrix metalloproteases.

Additional possibilities include agents that degrade the external structure of the vessel to permit expansion, such as collagenases, elastases, metalloproteinases; agents that promote inflammation that results in the release of factors the promote vessel expansion, such as interleukins 1, and TNF-alpha; and agents that block the effects of anti-inflammatory agents, such as blockers of IL-4, IL-10, IL-13, which are anti-inflammatory cytokines.

Referring to FIGS. 2C, 2B and 3B, in some embodiments when positioned over the fistula the shell edges need to be held together, particularly the edges of the portions 12a, 12b and the lower edges 17a of the portion 15. In other embodiments these edges of the shell 10 may not need a structure or fastener to hold edges together such as when the hinge 13 is constructed so that it can be spring-biased to a closed position.

Following the step illustrated in FIG. 3B the edges may be held together by a glue, stitching or magnets, including Bioerodible magnets (iron or cobolt flakes in erodible matrix) on both portions 10a, 10b or only one portion. The metals to be used are biocompatible and whose oxidation products and oxidation processes are biocompatible. Biodegradable metals are alloys of magnesium, zinc and iron. Of these, only iron can be magnetic. Forms of iron which can make a degradable magnet are:

Pure iron, martensitic; Carbonyl iron; Maghemite (Fe2O3); Magnetite (Fe3O4); Ferromagnetic iron; Ferrimagnetic iron; Superparamagnetic iron; and Iron oxide.

In some embodiments the shell 10 has eyelets that permit attachment of the shell 10 to adjacent tissues. These could be eyelets, loops, flanges, tabs or holes. A porous shell 10 would lend itself to direct suturing to the surrounding tissue.

According to some embodiments, the shell 10 is a hybrid where the shell is comprised of both bioresorbable polymer(s) and a durable polymer(s). The bioresorbable components hold the shell portion 15 at a smaller diameter which fits closely to the venous dimensions. As the bioresorbable portion degrades, the venous portion of the shell 10 easily dilates to a large diameter, which is set by the radial stiffness of the durable polymer component of the portion 15. According to one embodiment, the shell portion 15 is woven with both durable polymer filaments and bioresorbable polymer filaments where the durable ones are looser or can expand more once the bioresorbable filaments lose strength.

In general it is expected that the maturation process for the vein occurs within 12 weeks of forming the fistula. After this period, it is desirable to have at least the venous portion of the shell 10 bio-resorb or degrade to cause a significant loss in radial strength or stiffness of the shell portion surrounding or in contact with the maturing vein. According to some embodiments the shell may be made from, or portions thereof made from or strengthened by threads of the following material:
Poly(D,L-lactide-co-glycolide) (PLGA). This polymer comes in different ratios of D,L-lactide to glycolide. Preferred ratios are 50/50, 75/25, and 85/15
Poly(L-lactide-co-glycolide). This polymer also comes in different ratios. Glycolide content needs to be at least 5-50% by weight.
Poly(dioxanone) This polymer loses strength fast enough.
Poly(caprolactone-co-glycolide). It is glycolide that produces a faster degradation. This polymer with at least 5-50% glycolide by weight.
Poly(trimethylene carbonate-co-glycolide)
Poly(glycolide)
Poly(D,L-lactide) with inherent viscosity<0.6 dL/gm
Poly(L-lactide) with residual monomer content 0.2% (w/w)

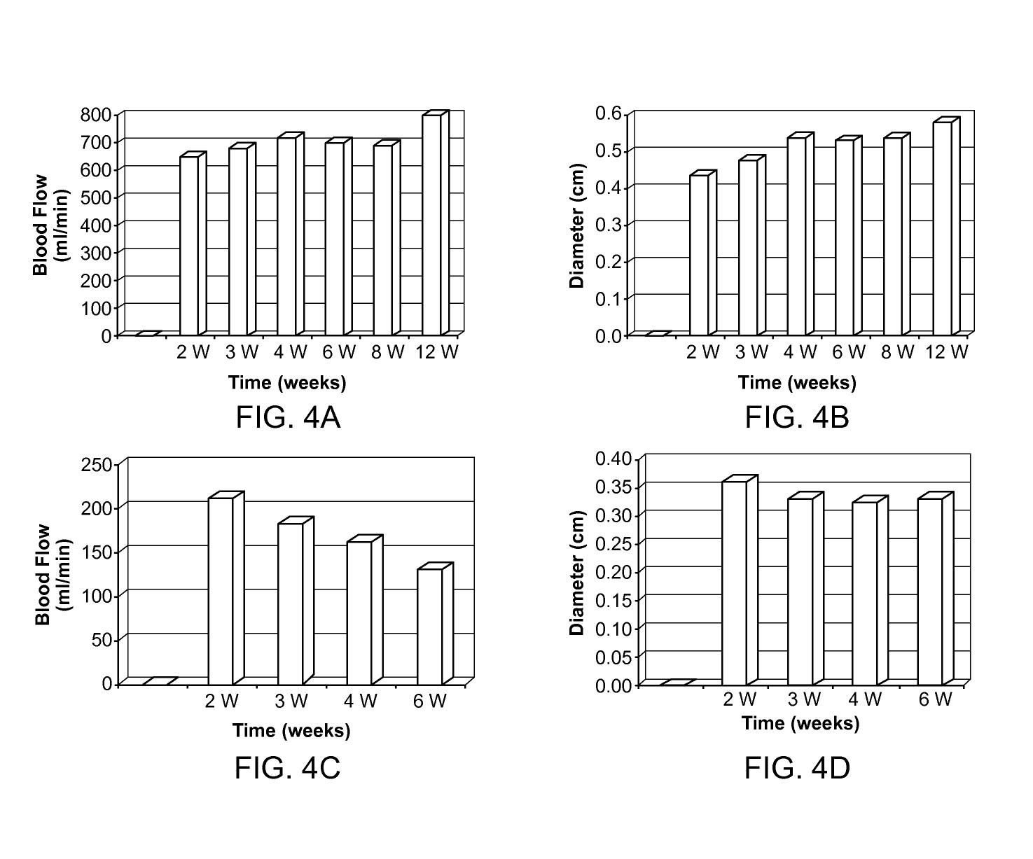

FIGS. 4A, 4B, 4C and 4D summarize the time course of fistula maturation. FIGS. 4A and 4B illustrate the blood flow rate and change in diameter for a successful AV fistula. FIGS. 4C and 4D illustrate the blood flow rate and change in diameter for a failing AV fistula.

There is an average venous growth rate of 0.2 mm/week (Corpataus). As such, according to some embodiments the shell 10 is capable of allowing the vein to expand at this rate through loss of mechanical properties or through loss of mass. A rapidly degrading polymer such as PLGA with low mass is presently preferred. TABLE 1 below summarizes the properties of a vein over a 12-week period following formation of the fistula.

TABLE 1
Remodeling of the forearm vein in Brescia-Cimino
hemodialysis access

| | | | | | Vein Wall |
|---|---|---|---|---|---|
| | | | | | Cross- |
| | Arterial | Venous | Blood | Shear | sectional |
| Time | diameter | diameter | flow | stress | Area |
| (weeks) | (mm) | (mm) | (ml/mm) | (dynes/cm²) | (dynes/cm²) |
| 1 | 3.275 | 4.430 | 539 | 24.5 | 4.4 |
| 4 | 3.555 | 5.041 | 640 | 18.1 | 5.3 |
| 12 | 3.310 | 6.620 | 750 | 10.4 | 6.9 |

Brescia-Cimino hemodialysis access is the standard surgical technique used to make arteriovenous fistulas. See, e.g., Bagolan P¹, Spagnoli A, Ciprandi G, Picca S, Leozappa G, Nahom A, Trucchi A, Rizzoni G, Fabbrini G., A ten-year experience of Brescia-Cimino arteriovenous fistula in children: technical evolution and refinements. J 1998 April; 27(4):640-4.

Referring again to FIGS. 2A, 2B, 2C, 3A and 3B, in some embodiments the shell 10 is pre-made to take the shape shown, e.g., pre-made as a wire or woven sleeve, or interconnected tubular bodies of a porous material. It will be appreciated that one disadvantage to this manner of providing the shell 10 is several different sizes may be need to accommodate differently-sized vasculatures.

In some embodiments the shell 10 is instead formed from a compound that can be shaped as needed for the patient's vasculature at the time of the procedure. To achieve this end, there is a provided a sheet of biocompatible material that achieves cross-linking (to form a permanent shape) by moisture cure, temperature, UV curing etc. The possible materials include epoxy, urethane butylcyanoacrylate, Bioglue etc. The material may be permanent or durable, or the material may be bioresorbable. Once shaped and placed around the vasculature, the ends may be held together by magnets, as disclosed earlier.

In some embodiments the moldable sheet is thermoset. The sheet is stored in a freezer. The surgeon would remove it from the freezer and immediately begin to form it around the artery/vein shaping it and cutting it to fit. As the material warms, a crosslinking reaction takes place which hardens the wrap. There are many chemistries capable of accomplishing this process, including thiol or amine/N-hydroxysuccinimide (NHS), thiol/maleimide, amine/thioester, sulfhydryl/vinyl sulfone, thiol/acrylate, thiol/vinyl ether, thiol/allyl ether, thiol/thiol, and biotin/avidin.

The sheet (or wrap) is monolithic meaning it is not porous. A thermoset system is one in which chemical cross linking occurs. This transforms the sheet from something that is flexible and moldable to one which is rigid, or at least has a memory for a certain shape. The crosslinking chemistry needs to be rapid, selective and biocompatible. Crosslinking systems typically have at least two components, i.e., component A and component B, although more than two is possible. It is preferred for the blended A and B components to have a glass transition temperature (TG) less than 40° C. in the hydrated state, using a simple part A and part B system as an example. Each part is composed of prepolymer molecules with a specific chemistry on the polymer chains.

Part A has one type of chemical functional group, which will be called “A groups,” and part B will have “B groups.” The specific A or B group chemistry can be at the ends of the polymer chains or arranged along the backbone, depending from the polymer chains. The two parts A and B are blended by the manufacturer either at low temperature and/or rapidly. During storage, the sheet is frozen or kept cold to prevent the crosslinking reaction. TABLE 2 lists some types of chemistries for Groups A and B, which are suitable for use in the body.

TABLE 2

| A Group | B Group |
|---|---|
| Primary, or | N-hydroxysuccinimide |
| secondary amine | |
| Thiol | Maleimide |
| Primary or | Thioester |
| secondary amine | |
| Sulfhydryl | Vinyl Sulfone |
| Thiol | Alpha-beta unsaturated ketone |
| Thiol | Vinyl Ether |
| Thiol | Allyl Ether |
| Thiol | Thiol (these groups from a cross link via oxidation |
| | of the two Thiol groups to form a disulphide bond) |
| Biotin | Avidin (this is not a chemical cross link but |
| | complexation) |

These pairs of A and B groups will react with each other to from a cross link. The A and B groups may be attached to a durable or biodegradable backbone polymer. A list of useful durable polymer backbones includes silicone, polyethylene, polypropylene, polybutylene, polyisobutylene and ethylene-alphaolefin copolymers, polyvinyl chloride, polyvinyl methyl ether, polyvinylidene chloride, polyvinyl acetate, ethylene-methyl methacrylate copolymers, ethylene-vinyl acetate copolymers, poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), polyurethane, cellulose acetate, cellulose butyrate, cellulose acetate butyrate, cellophane, cellulose nitrate, cellulose propionate, cellulose ethers, carboxymethyl cellulose, poly(ethylene glycol) (PEG), poly(ethylene oxide), poly(propylene oxide), poly(ether ester), polymers and co-polymers of 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethyl-phosphorylcholine (MPC) and n-vinyl pyrrolidone (VP), methacrylic acid (MA), acrylic acid (AA), poly(styrene-isoprene-styrene)-PEG (SIS-PEG), polystyrene-PEG, polyisobutylene-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), polyvinylidene fluoride, and poly(vinylidene fluoride-co-hexafluoropropylene).

A useful list of biodegradable polymers to which the A and B groups could be attached are collagen, gelatin, chitosan, alginate, fibrin, fibrinogen, starch, dextran, dextrin, hyaluronic acid, heparin, elastin, polyanhydrides, polyorthoesters, polyamino acids, poly(ester-amides), polyhydroxyalkanoates, poly(ester amides), polycaprolactone, poly(L-lactide), poly(D,L-lactide), poly(D,L-lactide-co-PEG) block copolymers, poly(D,L-lactide-co-trimethylene carbonate), polyglycolide, poly(lactide-co-glycolide), polydioxanone (PDS), polyorthoester, polyanhydride, poly(glycolic acid-co-trimethylene carbonate), poly(amino acids), poly(3-hydroxybutyrate) (PHB), poly(3-hydroxybutyrate-co-valerate) (PHBV), poly(3-hydroxyproprionate) (PHP), poly(3-hydroxyhexanoate) (PHH), poly(4-hydroxybutyrate), poly(4-hydroxyvalerate), poly(4-hydroxyhexanoate), poly(hydroxyvalerate), poly(tyrosine carbonates), poly(tyrosine arylates), poly(ester amide), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote), poly(4-hydroxybutyrate), poly(4-hydroxyvalerate), poly(4-hydroxyhexanote), poly(4-hydroxyheptanoate), poly(4-hydroxyoctanoate), poly(D,L-lactide), poly(D,L-lactide-co-caprolactone), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates), poly(tyrosine esters), poly(imino carbonate), and poly(glycolic acid-co-trimethylene carbonate).

For the thermoset sheet or wrap, after removal from the freezer, the surgeon would cut the wrap to shape (if needed) and then quickly place it around the artery/vein and anastomosis.

According to another embodiment the shell 10 is shaped from a porous sheet of material stored dry. In this embodiment, the sheet material is porous. The average pore size may be from 0.1 micron to 10 microns. Porous sheets can be made by lyophilization, leaching out of a porogen, a foaming process with an inert gas, mechanically by punching or drilling, or by lasing. Several chemistries are available that are responsive to moisture. Two preferred ones are isocyanate groups and cyanoacrylate groups. These chemistries don't require a Part A and Part B component. It would simply be one component with these groups attached (either all isocyanate or all cyanoacrylate). During storage, the sheet would be packaged in an impermeable package of some sort, preferably in an inert atmosphere. The physician would open the package, exposing the sheet to moisture. Exposure to moisture causes the groups to become reactive and initiates the cross linking reaction. This moisture can come from the ambient humidity, or exposure to biological fluids when implanted. The list of durable and biodegradable polymers to which the isocyanate or cyanoacrylate groups could be attached is the same as in the embodiment above.

According to another embodiment the shell 10 is made from a thermoplastic sheet (e.g. pebax, nylon, polyester, PLLA mesh) that is heated in a microwave then shaped around the artery when soft. The softening temperature would be above 37° C. but otherwise minimized or luminally insulated to minimize any thermal artery damage.

Thermoplastic polymers are those which can be processed via melt processing to form useful articles. They typically have a TG or melting point above ambient temperature. According to the embodiments a sheet or wrap could be stored at ambient temperature and the material can be monolithic or porous. It would be heated to temperature above 37° C., but when implanted in the body around the artery and vein, the temperature cannot be greater than about 47° C. Useful material would be a biocompatible material with a TG or melting point in the range of 37° C. to 50° C. Specific material which could achieve this are poly(ethylene-co-vinyl acetate), poly(ethylene-co-butyl methacrylate), poly(L-lactide-co-caprolactone), poly(L-lactide-co-trimethylene carbonate), poly(glycolide-co-caprolactone), and poly(glyolide-co-trimethylene carbonate).

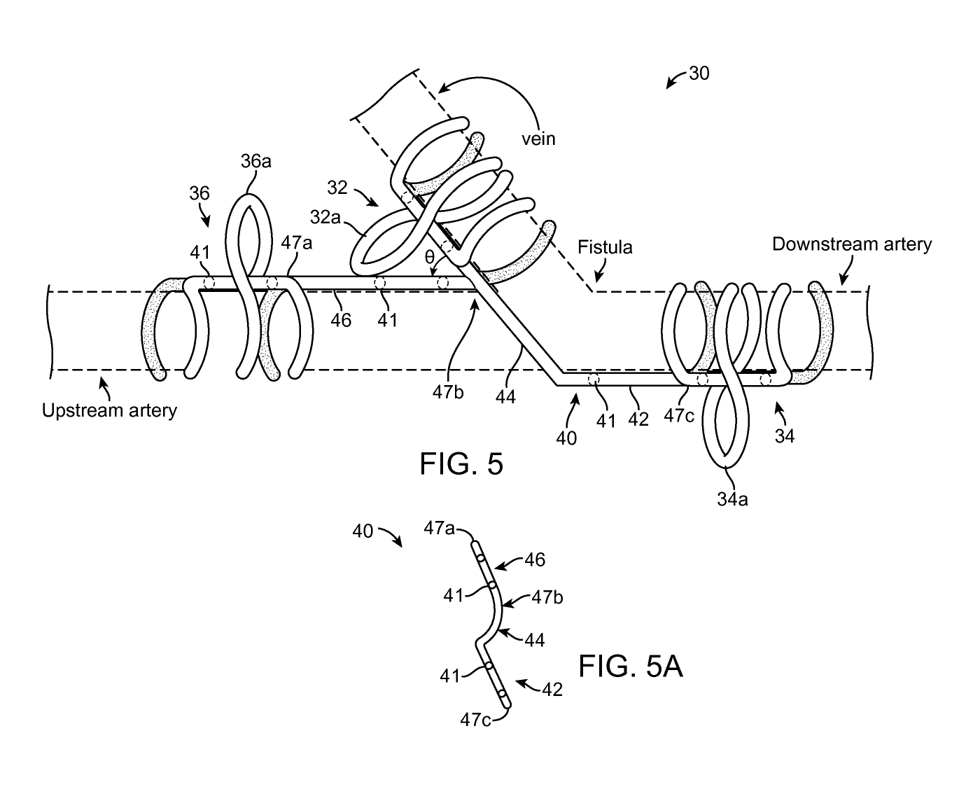

According to another aspect of the disclosure there are flexible and interconnected wire clips configured to close around venous and arterial portions adjacent the fistula to support and maintain the take-off angle θ. For example, referring to FIG. 5 there is shown an extravascular device 30 including three clips 32, 34 and 36 interconnected by a wire 40 shaped to set the angle θ between a venous clip 32 and upstream and downstream arterial clips 36 and 34, respectively. In some embodiments only the upstream arterial clip 36 is used (the downstream clip 34 is not included). Each of the clips of the device 30 would function similar to a clam shell in that arms wrap around the artery and/or vein. The clips 32, 34 and 36 may be made from an elastic wire, such as nitinol. After the anastomosis is made, the device 30 is secured as follows. Each of the clips 32, 34, 36 are held in an open state and placed around the artery and vein portions, respectively. The clips are released when in their desired location. The connecting wire portion 40, which connects the clips to each other, holds the clips at their respective orientations, thereby setting the take-off angle θ between the vein and artery. Glue 41 is applied to hold the device 30 at the fistula.

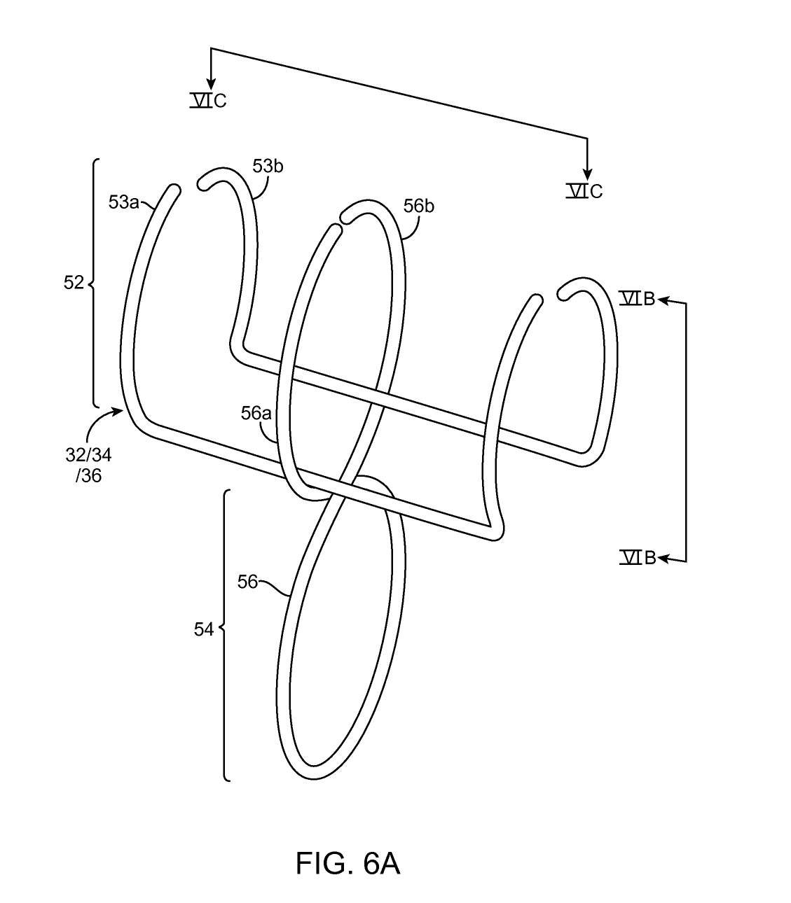

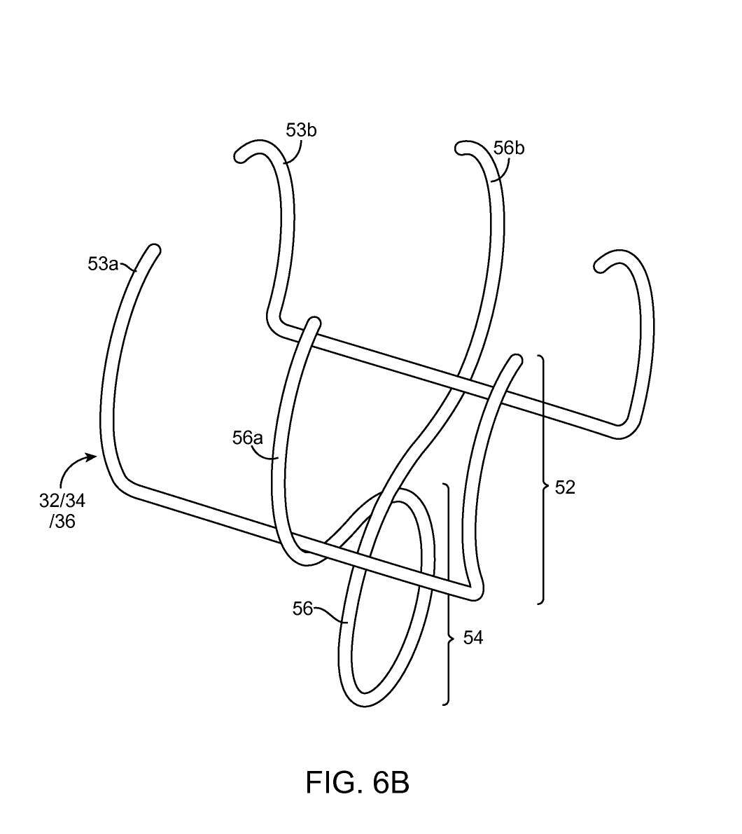

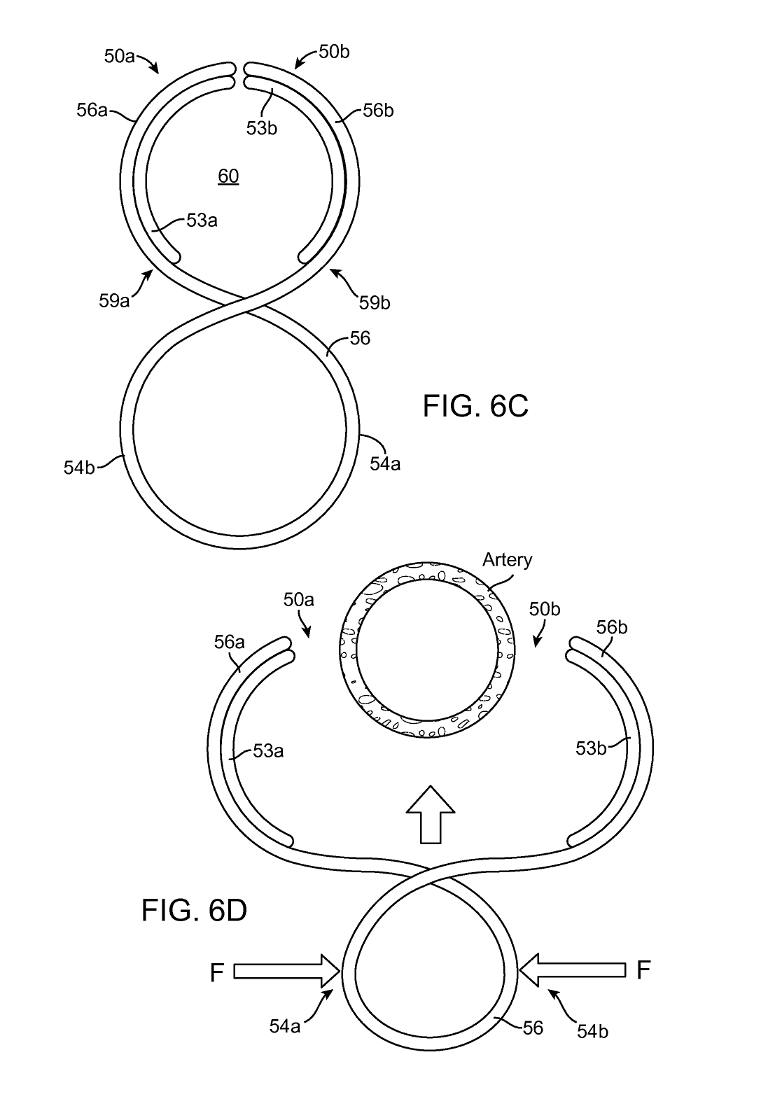

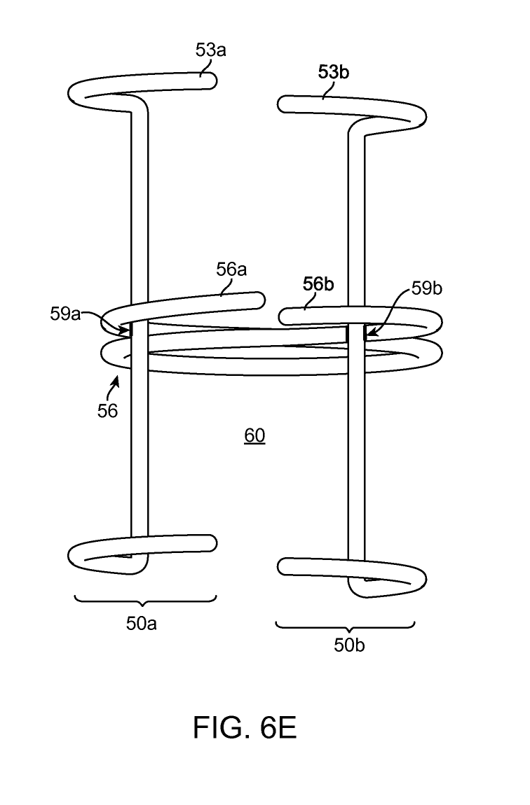

A benefit to having a spring clip, e.g., the clip illustrated in FIGS. 6A-6D and described in greater detail below, is that it gives a constant expansion force to the vein. Over time this radial force may mature to the desired diameter, i.e., increase as the vein diameter increases. As for manufacture, stamping, electrochemical, or an EDM cutting process followed by a coining (forming process) with a required heat set may be used, or the clips may be made from drawn nitinol tubing, which laser cut and cleaned to make the clips. A non-superelastic metal or plastic (e.g. non-nitinol) version may work similarly but would have lower allowable stresses and thus a more reduced range.

The clips 32, 34 and 36 may be made from either a round or flat cross section wire. High yield and elastic material such as 300 Series Stainless may be used. The thickness of the wire (narrowest dimension for a flat) would be 250 um to 2000 um wire. Possible materials would include 304 Stainless Steel, 316 Stainless Steel, Chrome Vanadium ASTM A231 or Chrome Silicon ASTM A 401. According to some embodiments the material has an Elastic Modulus of at least 180 MPa with a Tensile Strength of at least 750 MPa.

Referring to FIGS. 6A-6E there is shown various views of any of the interconnected clips 32, 34 or 36 from FIG. 5. According to the illustrated embodiment the clip 32, 34 or 36 is made from three pieces of wire 53a, 53b and 56 welded together to form a Trident-like structure, as depicted in FIG. 6A. Referring to FIG. 6E, which is a view taken from section VIC-VIC in FIG. 6A, and FIGS. 6B-6D, the clip has two deflectable arms, designated 50a and 50b and adapted to move away and towards each other when a pinching force is applied to lower portions 54a, 54b of wire 56 and released, respectively (FIG. 6D and FIG. 6B). Each arm 50a, 50b describes a half circle when the clip is viewed from the side (FIG. 6C). When the arms 50a, 50b are brought together they surround the vessel. The space between the arms 50a, 50b for the vessel is designated in the drawings as 60. The portions of the clip that close around the vessel are upper portions 56a, 56b of wire 56 and wires 53a and 53b.

Referring to FIGS. 6C and 6D, the lower portions 54a, 54b of wire 56, which serve as the deflection pieces for opening and closing the clip, form with the upper portions 56a, 56b of wire 56 a “figure eight” shape. The wires 53a, 53b are shaped to match the curvature of 56a, 56b portions. Each of the wires 53a and 53b are U-shaped. The wires 53a and 53b are welded to wire portions 56a, 56b at the locations 59a and 59b.

As depicted in FIG. 6D, when an inwardly-directed pinching force F is applied to the deflection portions 54a, 54b of wire 56 as shown, the arms 50a, 50b move away from each other sufficiently to allow passage of the vessel, e.g., artery, between the arms 50a 50b. The applied force may be finger pressure. When the vessel is between the arms 50a, 50b it occupies the space 60. The force is then removed. This causes the arms 50a, 50b to move back towards each other and surround the vessel (FIGS. 6A, 6C and 6E show the arms 50a, 50b position when no pinch force is applied).

Referring again to FIG. 5, in this embodiment there are three such clips 32, 34 and 36, as mentioned earlier, but in some embodiments there is no downstream clip 34 used. The wire 40 interconnects the clips 32, 34 and 36. The wire 40 has portions 42, 44 and 46. The portion 44 is curved, so that it extends around the vessel outer wall, e.g., in a semi-circular fashion. With this arrangement the deflection portions 36a, 34a may be disposed on opposites sides of the artery, which can aide in attachment. The portion 44 connects the portions 42 and 46, which are connected to the upstream and downstream arterial clips 36, 34.

FIG. 5A shows the shape of the wire 40. The portions 42, 44 can be essentially straight wire. The upstream arterial clip 36 is attached, e.g., by welding, at location 47a. The downstream arterial clip 34 is attached, e.g., by welding, at location 47c. The vein clip 32 is attached at location 47b. This connection is made so that the vein clip 32 is orientated at the take-off angle θ with respect to clips 34, 36.

In FIGS. 5 and 5A the locations designated as 41 on the wire 40 are locations for applying an adhesive which holds the device 30 to the vessel outer walls.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the claims should not be construed to limit the invention to the specific embodiments disclosed in the specification. Rather, the scope of the invention is to be determined entirely by the claims, which are to be construed in accordance with established doctrines of claim interpretation.

What is claimed is:

1. An apparatus, comprising:
a first tubular member having ends; and
a second tubular member connected to the first tubular member between the ends and extending from the second tubular member at a take-off angle θ;
wherein θ is less than about π/4 radians.

2. The apparatus of claim 1, wherein the apparatus is an extravascular arteriovenous (AV) wrap configured for supporting the venous portion of the fistula at the take-off angle.

3. The apparatus of claim 2, wherein the AV wrap comprises a porous body made from a biocompatible polymer, polymer blend, metal, metal alloy, or a combination thereof.

4. The apparatus of claim 3, wherein at least the second tubular member comprises both durable and biodegradable/bioresorbable polymers.

5. The apparatus of claim 4, wherein the second tubular member comprises durable polymer filaments and woven biodegradable or bioresorbable threads.

6. The apparatus of claim 2, wherein the AV wrap comprises one or more of a), b), c) and d):
e) an agent for preventing cell infiltration or to control neointimal hyperplasia, the agent being selected from the set consisting of everolimus, zotarolimus, ABT-578, sirolimus, umirolimus, biolimus, merilumus, myolimus, novolimus, temsirolimus, deforolimus, and AP23573;
f) an agent to encourage cell infiltration or to integrate a blood vessel with the AV wrap, the agent being selected from the set consisting of fibroblastic growth factor (FGF), basic FGF, platelet derived growth factor (PDGF), insulin like growth factor 1 (IGF-1), epidermal growth factor (EGF), granulocyte macrophage colony stimulating factor (GMCSF), human growth hormone (HGH), IL-1, TGF-B, and matrix metalloproteinases;
g) a vasodilator that promotes venous dilation, the vasodilator being selected from the set consisting of a nitric oxide donor, nitroglycerine, papaverine, calcium channel blocker, adenosine, prostacyclin, epinephrine, prostaglandin, L-arginine, bradykinin, natriuretic peptides, alpha blockers, adenocard, sodium nitroprusside, and matrix metalloproteases; and/or
h) an agent that degrades external structure of a vessel circumscribed by the AV wrap, the agent being selected from the set consisting of collagenases, elastases, metalloproteinases; agents that promote inflammation that results in the release of factors the promote vessel expansion, such as interleukins 1, and TNF-alpha; and agents that block the effects of anti-inflammatory agents, such as blockers of IL-4, IL-10, IL-13, which are anti-inflammatory cytokines.

7. The apparatus of claim 1, wherein the apparatus is a shell, the shell comprising:
a first part including a first edge,
a second part including a second edge, and
a hinge portion connecting the first and second parts,
wherein the edges are joined by a fastener.

8. The apparatus of claim 7, wherein the fastener is a magnet.

9. The apparatus of claim 7, wherein the hinge is a living hinge.

10. The apparatus of claim 1, wherein the apparatus comprises a porous body comprising a biocompatible polymer and/or metal.

11. The apparatus of claim 1, wherein the second tubular member is cylindrical or frustoconical.

12. The apparatus of claim 1, wherein the second tubular member has a first opening proximal the connection to the first tubular member and a second opening distal therefrom, wherein the second opening is larger than the first opening.

13. The apparatus of claim 1, wherein the second tubular member has a lower surface that forms with a lower surface of the first tubular member an angle of (180−θ) degrees, and the second tubular member has an upper surface that forms with an upper surface of the first tubular member at an angle of less than or equal to θ.

14. The apparatus of claim 1, wherein an average lumen size for the first tubular member is greater than an average lumen size for the second tubular member.

15. An apparatus adapted for being molded into an extravascular wrap for supporting a take-off angle of a venous portion of an arteriovenous (AV) fistula, comprising:
a sheet of moldable material;
wherein the sheet comprises one of a thermoset, moisture-activated or thermoplastic material.

16. The sheet of claim 15, wherein the sheet comprises one of a backbone material, and connected to the backbone material is material comprising a first group and a second group of material that chemically react with each other to form the wrap.

17. The sheet of claim 15, wherein the backbone material is a biodegradable or durable polymer.

18. The sheet of claim 15, wherein the first and second groups are selected from the set consisting of one of the pairs of Group A and Group B material in TABLE 2.

19. An arteriovenous (AV) guide, comprising:
a clip defining a first tubular space having a first bore axis; and
a member connecting the clip to at least one tubular body defining a second bore axis such that the first bore axis is oriented at an angle of θ or (180−θ) with respect to the second bore axis;
wherein θ is less than about π/4 radians.

20. The guide of claim 19, further including any combination of one or more of the following:
wherein the at least one tubular body is a second clip;
a pair of curved portions movable between an open position and a closed portion by deformation of a deflection portion interconnecting the curved portions, wherein when in the closed position the pair of curved portions surround the first tubular space;
wherein the clip is formed by a first and second u-shaped wire welded to a serpentine wire, the serpentine wire comprising the deflection portion; and
wherein the first tubular space is larger than a second tubular space circumscribed by the tubular body.
